(19) United States
(12) Patent Application Publication
(10) Pub. No.: US 2021/0186754 A1
(43) Pub. Date: Jun. 24, 2021

(54) MINIMALLY INVASIVE GLAUCOMA SURGERY DEVICES, SYSTEMS, AND ASSOCIATED METHODS

(71) Applicant: MICROSURGICAL TECHNOLOGY, Redmond, WA (US)

(72) Inventors: David Michael Colvard, Encino, CA (US); Bibianna A. Cha, Seattle, WA (US); Kurt Faulhaber, Renton, WA (US); Robert Raney, Clyde Hill, WA (US)

(21) Appl. No.: 17/257,841

(22) PCT Filed: May 25, 2018
(86) PCT No.: PCT/US18/34783

§ 371 Date: Jan. 4, 2021

Related U.S. Application Data
(60) Provisional application No. 62/511,887, filed on May 26, 2017.

(51) Int. Cl. A61F 9/009

(52) U.S. Cl. CPC A61F 9/009; A61F 2009/0052

(57) ABSTRACT

Disclosed are systems, devices, and methods of gonioprism docking with ocular surfaces using vacuum seals for improved conditions during medical procedures on ocular surfaces.

### CROSS REFERENCE TO RELATED APPLICATIONS

This application is a U.S. National Stage under 35 USC 371 of International Patent Application No. PCT/US2018/034783, filed May 25, 2018, which claims priority to U.S. Provisional Patent Application No. 62/511,887, filed May 26, 2017, the disclosures of both which are hereby incorporated in their entireties by reference.

### FIELD OF THE INVENTION

The subject matter described herein relates generally to systems, methods, and devices for maintaining a gonioprism in a fixed position with respect to an eye during a surgical or other procedure.

### BACKGROUND OF THE INVENTION

Currently, there is a great deal of clinical interest, including research and development, in the use of very small, intraocularly implantable devices for the treatment of glaucoma. These devices generally fall into a particular category of devices and are collectively referred to as Minimally Invasive Glaucoma Surgery (MIGS) devices. Presently, at least four devices have been approved for use by the United States Food and Drug Administration. The first is the iStent, which is manufacturer by Glaukos and is placed in the ocular trabecular meshwork. The second is the Cy-pass, which is manufactured by Alcon and is placed in the ocular supra-choroidal space. The third is the iTrack, manufactured by Ellex, which is a micro-catheter used to dilate Schlemm's canal. The fourth is the Xen Gel Stent, manufactured by Allergan, that helps to create a filtration pathway from the anterior chamber, through the sclera, and into the subconjunctival space. Each of these devices is designed to improve aqueous fluid out-flow and to reduce intraocular pressure. These devices are surgically placed in an area within the eye called “the angle.”

The angle is an area located within the anterior chamber of the eye where the cornea and the iris join 360 degrees around the periphery of the iris and cornea. This area of the anterior chamber is located under the peripheral corneal area and cannot be seen or otherwise visualized by looking directly at the eye. Therefore, in order to visualize the angle, a physician must be able to look around this peripheral corneal area, similar to looking around a corner. Devices that have been employed to perform this action include small hand held optical prisms, referred to as gonioprisms.

Gonioprisms are devices that are used during medical procedures, especially on the eye, to view obscured or hidden structures by providing angular views around intermediate anatomical structures. They generally provide a field of view of anterior ocular chamber structures and anterior chamber angles during procedures that provide for implantation of devices, application of lasers, and other surgical manipulation of structures in the eye, including goniotomy. Gonioprisms must be correctly positioned for effective use. Various examples of prior art gonioprism positioning tools have been developed and most of these require that they be held by hand during a surgical procedure, usually by the surgeon. These tools are usually held in the surgeon's hand and must be maintained in a particular position to correctly view the desired structures.

While some gonioprism positioning tools include extensions, flanges, handles, or other structures designed to help maintain position with the ocular globe during surgical procedures, they can be unwieldy and may introduce an increased surgeon manipulation of the device to achieve the desired visualization effects. Additionally, most of these tools require the surgeon to maintain a particular amount of contact pressure with the patient's cornea or other ocular structures, which can be challenging. Where contact pressure by the surgeon is too light, the interface between the gonioprism lens and the surface of the cornea may be lost, and the surgeon will no longer be able to see the desired location or structures. Where contact pressure by the surgeon is too great, the cornea may crinkle or fold into Descemet's membrane in the cornea, resulting in the surgeon no longer being able to see the desired location or structures. Even experts in the field that specialize in these types of ocular surgery procedures may struggle with positioning related challenges. Poor visualization as a result of positioning problems is known to be one of the primary impediments to successful ocular surgery procedures.

A number of examples of pertinent prior art gonioprisms and positioning tools exist. One example is U.S. Pat. No. 7,125,119, which describes a standard gonioprism with a contact lens to fit on a cornea specifically for laser procedures like SLT. Another example is WIPO Publication No. 99/20171, which describes a contact lens with a vacuum to maintain contact with an eye surface for vitreoretinal surgery. This lens has an access port to allow the introduction of instruments into the posterior portion of the eye behind the lens, but the access port does not pass through a vacuum element area. WIPO Publication No. 92/07501 describes a contact lens that provides a wide field of vision for retinal ophthalmoscopy. U.S. Pat. No. 5,046,836 describes a contact lens for retinal indirect ophthalmoscopy. U.S. Pat. No. 5,200,773 describes a contact lens for retinal indirect ophthalmoscopy. U.S. Pat. No. 5,886,812 describes a contact lens connected to a microscope for retinal indirect ophthalmoscopy. U.S. Patent Pub. No. 2012/0257167A1 describes a hand-held gonioscope, including a prism with a handle. U.S. Pat. No. 8,070,290 describes another hand held gonioscope, including a prism with a handle. U.S. Pat. No. 7,419,262 describes yet another hand held gonioscope including a prism with a handle. Each of these prior art disclosures is incorporated herein by reference. However, each these devices lack the specific features and do not provide the benefits of the embodiments described herein.

It is therefore desirable to provide improved systems, devices, and methods that allow a gonioprism to maintain optimal positioning with respect to a corneal location by selectively applying a safe and effective amount of vacuum pressure to an external eye surface which can improve hands-free visualization of ocular structures through the gonioprism and allow access to structures within the anterior chamber.

### SUMMARY OF THE INVENTION

Disclosed are systems, devices, and methods that maintain optimal positioning of a gonioprism with respect to a corneal location. In various embodiments, this is achieved by selectively applying a safe and effective amount of vacuum pressure to an external eye surface and results in improved, hands-free visualization of ocular structures through the gonioprism.

These systems, devices, and methods include the use of vacuum docking of a gonioprism to an external eye surface that provides a removable fixation to the eye and allows a physician to accurately and effectively treat parts of the eye, including the cornea. In some embodiments, gonioprisms can be removably or detachably coupled with a vacuum dock, while in others, they may be fixedly coupled. Vacuum mechanisms can include active or passive pumping mechanisms, vacuum syringes including one or more valves, and others in various embodiments.

### BRIEF DESCRIPTION OF THE DRAWINGS

Illustrated in the accompanying drawing(s) is at least one of the best mode embodiments of the present invention.

FIG. 1A shows an example embodiment of an anatomical diagram of an eye cross section with a reference key.

FIG. 1B shows an example embodiment of an intracorneal angle diagram.

FIG. 2 shows an example embodiment of a prior art tool for maintaining a fixed position of a gonioprism.

FIGS. 3A-3B show an example embodiment of a prior art gonioprism.

FIG. 4 shows an example embodiment of a gonioprism separate from a removable vacuum docking device.

FIG. 5 shows an example embodiment of a gonioprism and removable vacuum docking device after being coupled.

FIG. 6 shows an example embodiment of a gonioprism and removable vacuum docking device coupled.

FIG. 7A shows a perspective view of an example embodiment of a gonioprism and vacuum docking device coupled together.

FIG. 7B shows a cross-sectional view of an example embodiment of a gonioprism and vacuum docking device coupled together.

FIG. 7C shows a bottom view of an example embodiment of a gonioprism and vacuum docking device coupled together.

FIG. 7D shows a perspective view of an example embodiment of a gonioprism and vacuum docking device coupled together.

FIG. 8A shows a perspective view of an example embodiment of an upright gonioprism and vacuum docking device coupled together.

FIGS. 8B and 8C show a cross-sectional view of an example embodiment of an upright gonioprism and vacuum docking device coupled together.

### DETAILED DESCRIPTION

Before the present subject matter is described in detail, it is to be understood that this disclosure is not limited to the particular embodiments described, as such may vary. It should also be understood that the terminology used herein is for the purpose of describing particular embodiments only, and is not intended to be limiting, since the scope of the present disclosure will be limited only by the appended claims.

Disclosed herein are systems, devices and methods for improved, hands-free visualization of intra-ocular structures through a gonioprism during medical procedures. In various embodiments, these can include standard, modified, or customized vacuum docks that, when coupled with gonioprisms and engaged, maintain a fixed position with respect to a coupled eye surface location. As such, they can remain fixed to the eye for delicate procedures in which physicians or surgeons would benefit from having full use of both hands without having to constantly maintain a gonioprism in position using one hand.

Example embodiments disclosed herein included gonioprisms that are removable or detachable using screwing, clamping, latching, or other mechanisms with a vacuum docking station. In various embodiments, gonioprism docking devices can include one or more disposable components. In some embodiments, these components can be reusable if properly sterilized.

In some embodiments, other docking functionality can also be included. This can be done in combination with or as substitution for vacuum docking functionality in various embodiments. To elaborate, mechanical docking with one or more mechanical structures can be provided for in some embodiments. This can include docking with a speculum, with sutures, with lighting components, with sensors, with measurement components, and with others, as appropriate. In some embodiments, this can be performed during a pre-procedure process, while in other embodiments, it can be performed during a procedure.

In some embodiments, systems, devices, and methods can include an apparatus that makes and maintains contact a portion of a patient's cornea and not another portion of the eye, and which provide access to the anterior chamber.

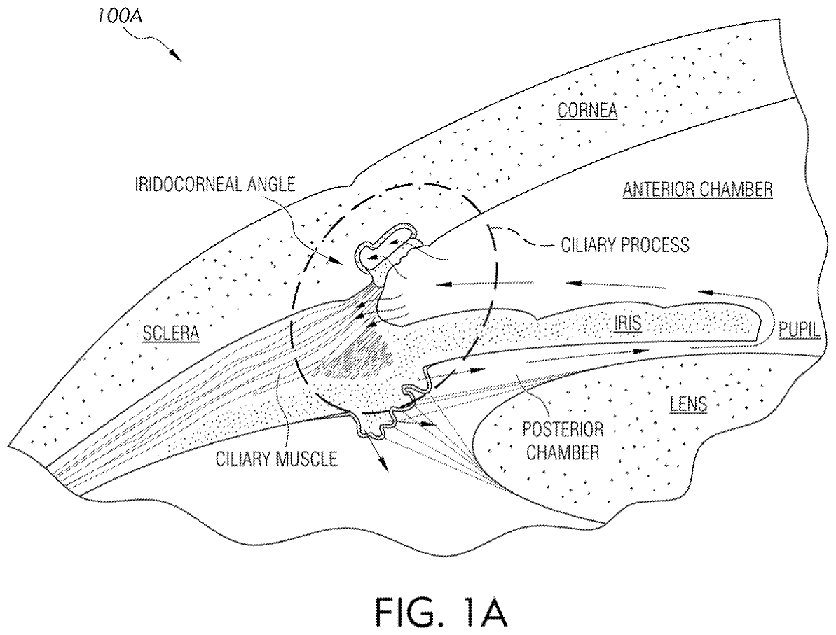

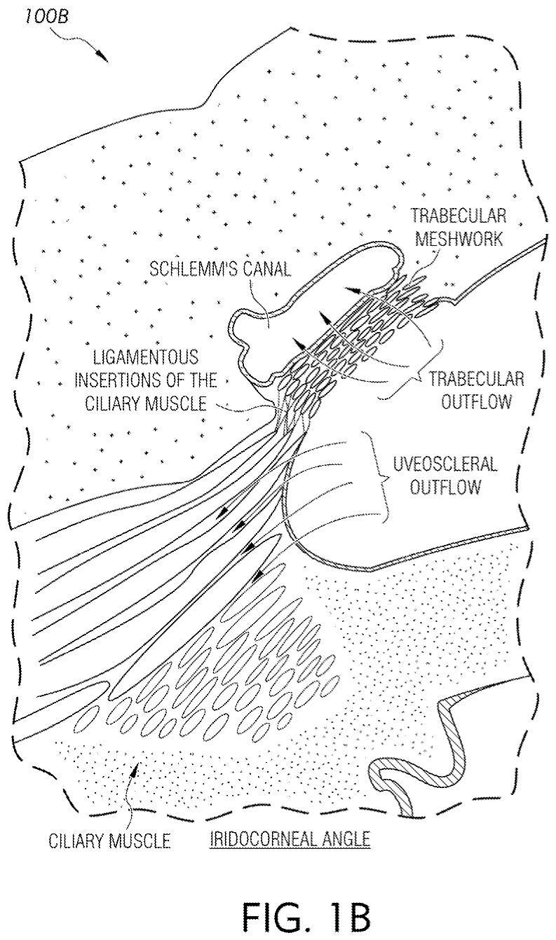

FIG. 1A shows an example embodiment of an eye anatomy cross-sectional diagram 100A showing a cornea and sclera interface. As shown in the example embodiment, a location where the cornea and sclera interface can include anatomical features including the iridocorneal angle.

FIG. 1B shows an example embodiment of an intracorneal angle diagram 100B. As shown in the example embodiment, Schlemm's canal can be located above the Trabecular meshwork and allow for Trabecular outflow. Ligamentous insertions of the ciliary muscled can be coupled with the Trabecular meshwork and uveoscleral outflow can occur between the anterior chamber and the ciliary muscle.

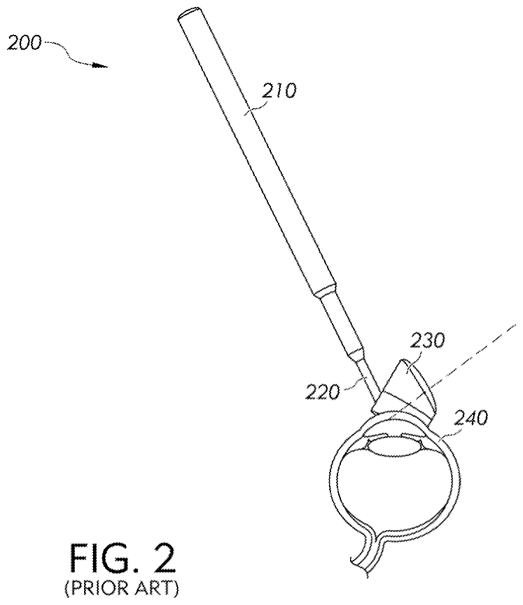

FIG. 2 shows an example embodiment of a prior art tool 200 for maintaining a fixed position of a gonioprism. As shown in the example embodiment, the tool can include a handle 210 that is coupled at a distal end 220 with a gonioprism 230. This can be held in position with an exterior ocular surface 240 to provide the advantages of viewing ocular structures through the gonioprism lens that would be otherwise hidden based on anatomical intraocular arrangements.

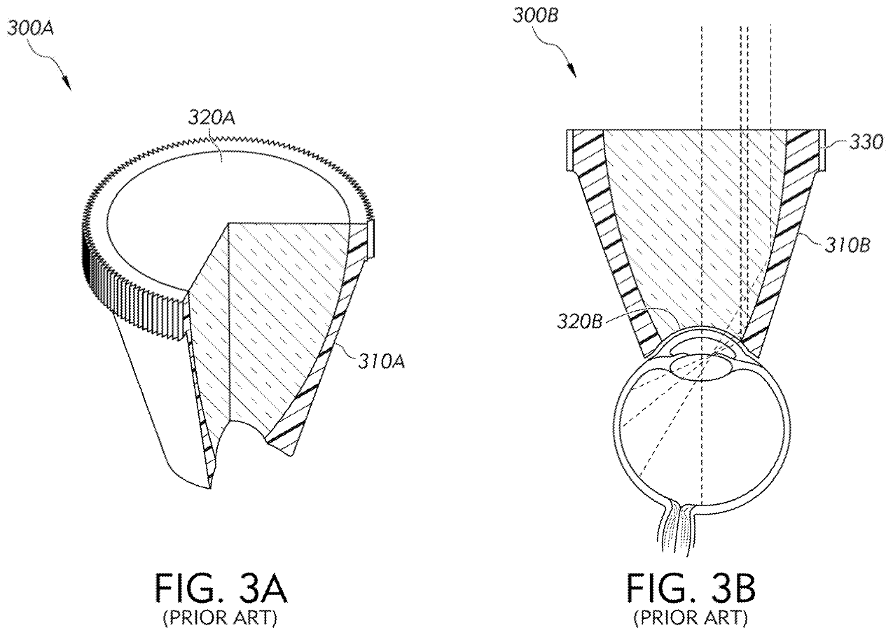

FIGS. 3A-3B show example embodiments 300A and 300B of a prior art gonioprism. As shown a proximal surface or lens 320A or 320B of the gonioprism 310A or 310B can be concave, convex, or flat, while a distal surface will generally be flat concave to accommodate the convex structure of the eye. The gonioprism 310A or 310B can be removably or permanently coupled with an exterior housing 330 that is opaque and does not allow light from the sides of the gonioprism to enter and interfere with the structures that are desired for viewing during a procedure.

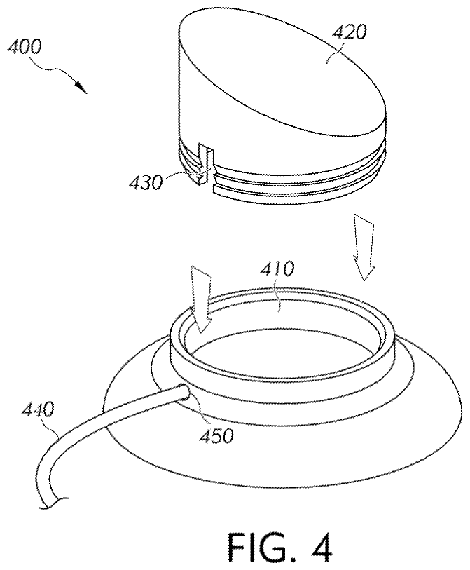

FIG. 4 shows an example embodiment 400 of a gonioprism 410 separate from a removable vacuum docking device 420. As shown in the example embodiment, a gonioprism 410 can be a steady state gonioprism, although in other embodiments, the gonioprism may have more than one state. This gonioprism 410 can be removably or permanently docked with the vacuum docking device 420 using a docking mechanism. This can be a screwing mechanism with grooves, a latching mechanism, or other mechanisms as appropriate. As such, removable docking 420 can be slidably engaged, rotatably engaged, or achieved using various other types of engagement based on component arrangement.

To elaborate, these mechanisms can generally be considered docking mechanisms, whereby a gonioprism is adapted for coupling with a docking device using the docking mechanism. These docking mechanisms can allow for docking before or during a procedure. Additionally, in some embodiments these docking mechanisms can allow for orientation and manipulation of docked gonioprisms to desired orientations after docking.

As shown, one or more vents or channels 430 in a portion of the gonioprism can allow ingress, egress, or both from ocular surfaces. For example, balanced salt solutions (“BSS”) that are used to help irritate the eye, saline solution, and other fluids can be moved through the vent 430 to assist in the procedure or allow natural fluid flow with respect to normal eye functioning. Thus, vents 430 can provide an interface with the vacuum docking device as a gonioprism is rotated or otherwise coupled into an operable position with respect to the vacuum docking device 420.

Also shown in the example embodiment is a vacuum hose 440. This hose 440 can be removable or permanently fixed to the vacuum docking device 410 at a vacuum hose interface 450 and, when a vacuum is coupled at a distal hose end, the proximal hose end will draw fluid, such as air, through the hose. This can operate to seal the gonioprism 420 to the docking device 410 in some embodiments. In some embodiments, it can operate to seal the docking device 420 and gonioprism 410 to an ocular surface.

Additionally, it should be understood that in various embodiments, seals can prevent fluid movement between one or more components, and may be slidably or otherwise engaged between components. For example, one or more rubber rings can be provided at the interface between the gonioprism and the vacuum docking device.

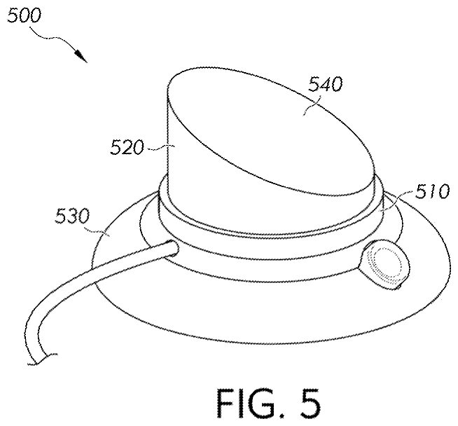

FIG. 5 shows an example embodiment 500 of a gonioprism 520 and removable vacuum docking device 510 after being coupled. As shown in the example embodiment, a skirt 530 can operably engage an eye surface and can be permanently or removably coupled with a docking device base. Docking device 510 base can be generally cylindrical and can have a hollow or solid interior space defined by a circumferential wall. Skirt 530 can be cone shaped with a narrower radius end near docking device base and wider radius end that terminates in a circumferential ring that can engage an ocular surface. Skirt 530 and base can be coupled at a hard-plastic ring to base using adhesives or other appropriate coupling mechanisms.

Skirt 530 can be an elastomeric material in various embodiments. As such it can be soft, pliable plastic, moderate plastic, or slightly harder plastic as appropriate. Gonioprisms 520 can include a lens 540 made of polished glass or molded and polished plastic material that is about 11 mm to about 12.5 mm or 13 mm. Vacuum docking device base can generally be a hard material shell that is operable to create and sustain a vacuum when engaged.

Vacuum docking device skirt 530 and base can be opaque in some embodiments, such that they do not allow light to penetrate through their surfaces. Gonioprism lens 540 is generally transparent and allows light to pass through a distal and proximal end in one or both direction in order to view the subject material below. Lighting in many embodiments is provided by a microscope, while in some embodiments, ambient lighting in the operation room can be sufficient. Also, in some embodiments, lighting mechanisms that are coupled with the docking device 510 or gonioprism 530 can be provided. These may include one or more lighting elements, such as LED's, that are powered using one or more power sources, such as removable or permanently attached batteries or power cables. It should be understood that on and off switches or buttons can allow for their associated effects.

In some embodiments, docking device skirt 530 can include a pre-operative treatment that aids in creating an effective vacuum seal, protects the ocular surface from damage, or performs some other functionality.

As shown in the example embodiment, the gonioprism 520 can detachably or removably couple with the vacuum docking device 510. The vacuum docking device 510 can include a vacuum skirt 530 that removably couples with at least a portion of a sclera of an eye for stability. Vacuums contemplated herein are generally operable to perform the functions described herein without requiring an excessive amount of vacuum pressure that may cause injury to the patient. Vacuum devices providing suction can be integrated with vacuum docking devices in some embodiments. Once a vacuum has been engaged, the vacuum docking device generally remains fixed with respect to its position with respect to its engaged eye surface. In some embodiments, the skirt and device can be slightly moved or repositioned, even after the vacuum has been engaged.

Although generally described herein are various vacuum docking mechanisms, other docking mechanisms can be provided in various embodiments. For example, additional mechanical docking with one or more mechanical structures can be provided for in some embodiments, including: docking with tools such as a speculum, with sutures, with lighting components, with sensors, with measurement components, and with others, as appropriate. In some embodiments, this can be performed during a pre-procedure process, while in other embodiments, it can be performed during a procedure.

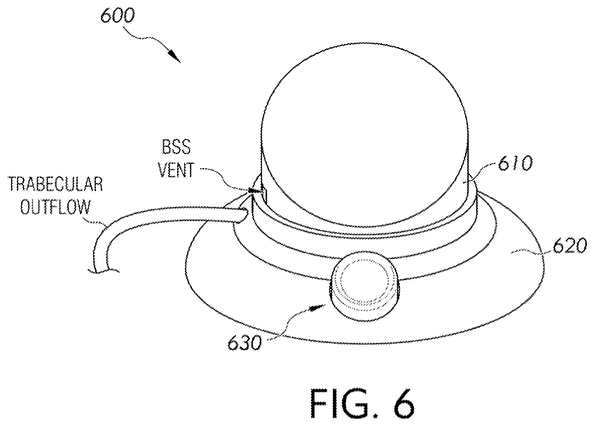

FIG. 6 shows an example embodiment 600 of a gonioprism 610 and removable vacuum docking device 620 coupled. As shown, the vacuum docking device 620 can include an opening or port 630 that extends through a portion of the docking device 620 and is defined and separated from an exterior area by at least one wall. In the example embodiment, this is a cylindrically shaped hole that can extend from a treatment surface of the eye to the area above the docking device 620 and defined by a circumferential wall. This opening 630 can allow access to the treatment surface at the corneal interface to allow access to a corneal incision location. with a vacuum seal. Here, the opening 630 is through the skirt, although it can also be through the docking device body in some embodiments. The opening 630 can be about 3 mm wide at its diameter in some embodiments with a depth or length of about 1.5 mm. The opening 630 is generally located at an anterior location of the skirt.

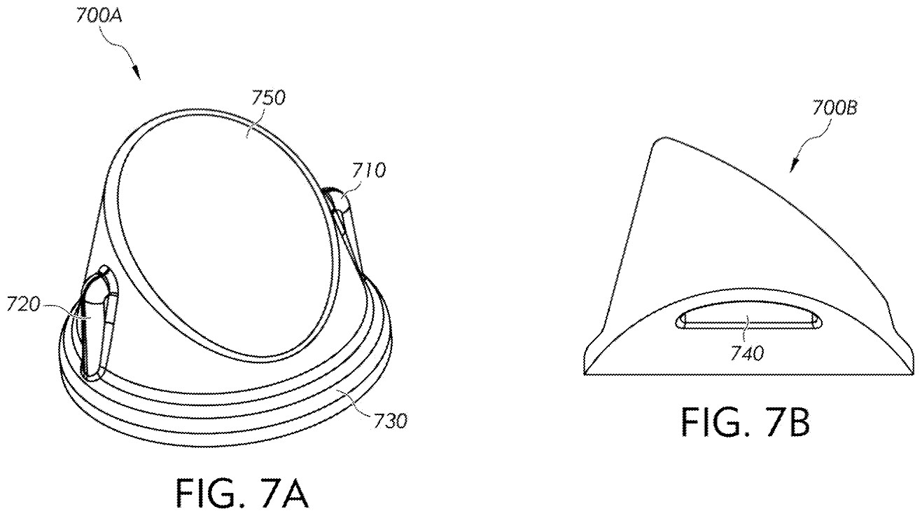

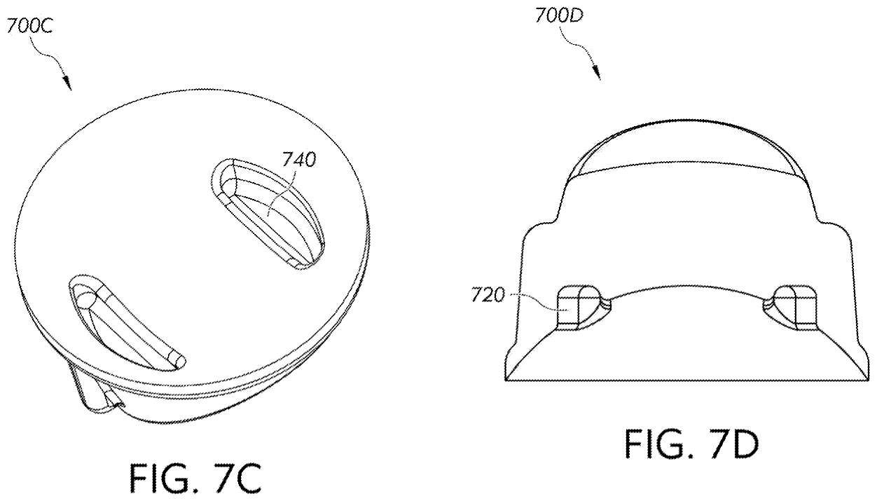

FIGS. 7A-7D illustrate various embodiments 700A, 700B, 700C, and 700D of a gonioprism 710 with a vacuum docking device 720 coupled that may be detachable from each other. As opposed to a vacuum hose, in some embodiments, the gonioprism 710 may have one or more baffles and one or more openings 740 at the bottom. The pressing of the baffles may create a vacuum within the skirt caused to the suction and expulsion of air through the bottom opening 740 that would allow it to adhere to one location as described above. The gonioprism 710 may comprise one or more push buttons 720 that may allow to break the vacuum seal so that the gonioprism may be adjusted to a different location. The gonioprism 710 may comprise one or more lens 750. The gonioprism 710 may include a single spherical radius that lays on the cornea. In some embodiments, the gonioprism 710 may have a port/opening, as described above, or any such possibility that would allow access to the anterior chamber. In some embodiments, the contact area of the gonioprism 710 and the skirt 730 may be small enough that no port/opening may be required. The gonioprism 710 and the skirt 730 may be a unitary piece or detachable.

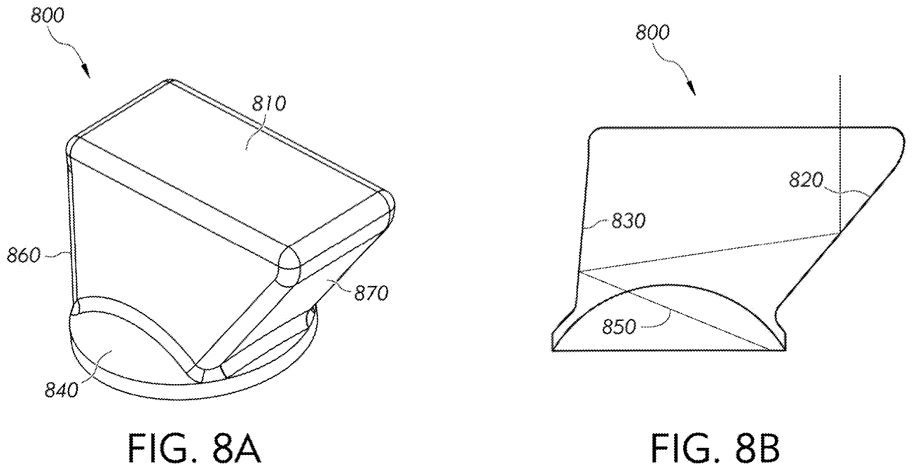

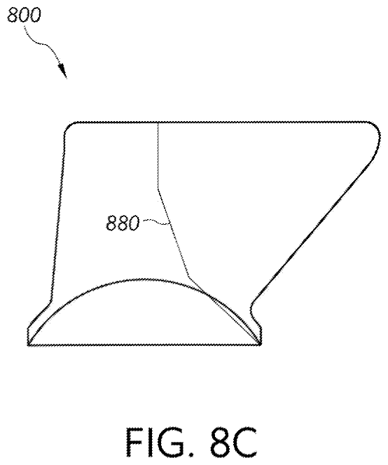

FIGS. 8A-8C illustrate a hands-free upright gonioprism 810 that allows for the user to look straight down without titling the patient's head or the microscope to see the Trabecular meshwork. The gonioprism 710 may be permanently or detachably attached to a skirt 840. The gonioprism 810 may have two or more mirrors 820 and 830 or optical stacks or any other mechanism on any location on the gonioprism 810 such as the walls 860 and 870 that may allow light 850 to refract or reflect in a way that the patient's head or the microscope need not be titled. The walls 860 and 870 may be conveniently tilted to achieve optimal visibility without having to tilt the microscope or the patient's head. The gonioprism 810 may have vacuum mechanisms as described in previous embodiments. In some embodiments, the gonioprism 810 may have a port/opening, as described above, or any such possibility that would allow access to the anterior chamber. In some embodiments, the contact area of the gonioprism 810 and the skirt 840 may be small enough that no port/opening may be required. As illustrated in FIG. 8C, the light 880 may refract/reflect through multiple optical material stacks or other mechanism on any location so that mirrors on the walls may not be needed for the surgeon to see the Trabecular meshwork. directly while the patient lays upright.

While the embodiments herein describe a gonioprism, the gonioprism may be replaced or supplemented by any other optical device, including but not limited to surgical contact lenses, Retinal Vitrectomy Lenses, Indirect Contact Surgical Lenses, Aspheric Macular Lenses. Additionally, the vacuum pressure (or other sealing pressures) being applied/exerted on both the skirt and the cornea, in some embodiments, the vacuum pressure (or other sealing pressures) may be applied/exerted to the skirt only and not the cornea to reduce pressure induced folds in the cornea which may tend to reduce visualization. Although shown as an port/opening in one or more components in the device, in some embodiments the port/opening may not be configured as shown in the figures. For example, in some embodiments the skirt may not be completely circumferential and instead may include one or more walls defining a triangular portion or slice where procedures can be executed. As such, a port can be defined by part of a discontinuation of a circumferential surface or wall. Thus, in some embodiments, ports do not involve nearby engagement of the vacuum docking system.

Additionally, in some embodiments a vacuum dock does not engage an entire area below a docking device or an entire area below a particular component of a docking device. For example, where a skirt is circumferential in nature, a vacuum pump and docking device configuration may not create a vacuum seal within the entire circumference of the skirt. Instead, it may engage an ocular surface at one or more specific points to create the vacuum seal and maintain device positioning using suction.

Although not shown in the example embodiment, in some embodiments automatic digital or analog vacuum gauges can be included that display vacuum pressure present within the interior of the docking device when in use. As such, these gauges can be coupled with and influenced by sensors, which are not shown.

As shown in the example embodiment, the gonioprism can be rotated with respect to the vacuum docking device, which, in some embodiments, can occur during a procedure. Although not shown, upward and downward or proximal and distal movement of the gonioprism can be actuated using a screw, lever, or other appropriate mechanism.

As used herein and in the appended claims, the singular forms “a”, “an”, and “the” include plural referents unless the context clearly dictates otherwise.

The publications discussed herein are provided solely for their disclosure prior to the filing date of the present application. Nothing herein is to be construed as an admission that the present disclosure is not entitled to antedate such publication by virtue of prior disclosure. Further, the dates of publication provided may be different from the actual publication dates which may need to be independently confirmed.

It should be noted that all features, elements, components, functions, and steps described with respect to any embodiment provided herein are intended to be freely combinable and substitutable with those from any other embodiment. If a certain feature, element, component, function, or step is described with respect to only one embodiment, then it should be understood that that feature, element, component, function, or step can be used with every other embodiment described herein unless explicitly stated otherwise. This paragraph therefore serves as antecedent basis and written support for the introduction of claims, at any time, that combine features, elements, components, functions, and steps from different embodiments, or that substitute features, elements, components, functions, and steps from one embodiment with those of another, even if the following description does not explicitly state, in a particular instance, that such combinations or substitutions are possible. It is explicitly acknowledged that express recitation of every possible combination and substitution is overly burdensome, especially given that the permissibility of each and every such combination and substitution will be readily recognized by those of ordinary skill in the art.

In many instances entities are described herein as being coupled to other entities. It should be understood that the terms “coupled” and “connected” (or any of their forms) are used interchangeably herein and, in both cases, are generic to the direct coupling of two entities (without any non-negligible (e.g., parasitic) intervening entities) and the indirect coupling of two entities (with one or more non-negligible intervening entities). Where entities are shown as being directly coupled together or described as coupled together without description of any intervening entity, it should be understood that those entities can be indirectly coupled together as well unless the context clearly dictates otherwise.

While the embodiments are susceptible to various modifications and alternative forms, specific examples thereof have been shown in the drawings and are herein described in detail. It should be understood, however, that these embodiments are not to be limited to the particular form disclosed, but to the contrary, these embodiments are to cover all modifications, equivalents, and alternatives falling within the spirit of the disclosure. Furthermore, any features, functions, steps, or elements of the embodiments may be recited in or added to the claims, as well as negative limitations that define the inventive scope of the claims by features, functions, steps, or elements that are not within that scope.

What is claimed is:

1. An improved ocular surgical docking system, comprising:
a docking base; and
a docking skirt coupled to a distal end of the docking base,
wherein the docking base is operable to be coupled with a gonioprism comprising proximal lens.

2. The improved ocular surgical docking system of claim 1, further comprising at least one access port through a portion of the docking system that provides access to an ocular surface during medical procedures.

3. The improved ocular surgical docking system of claim 1, further comprising a vacuum hose, operably coupled with the docking base.

4. The vacuum hose of claim 3, wherein the vacuum hose is operable to remove fluid from an interior volume of the docking skirt placed at a docking location, such that the skirt creates a vacuum seal at the docking location.

5. The improved ocular surgical docking system of claim 4, further comprising at least one access port through a portion of the docking system that provides access to an ocular surface during medical procedures.

6. The improved ocular surgical docking system of claim 4, wherein accessing the ocular surface during medical procedures through the at least one access port does not interfere with the docking base fixation with respect to the docking location.

7. The improved ocular surgical docking system of claim 1, wherein the gonioprism is operable to rotate with respect to the docking station.

8. The improved ocular surgical docking system of claim 1, wherein the gonioprism is permanently coupled with the docking base.

9. The improved ocular surgical docking system of claim 1, wherein the proximal lens is concave.

10. The improved ocular surgical docking system of claim 1, wherein the gonioprism is coupled with an exterior housing that is opaque.

11. The improved ocular surgical docking system of claim 1, wherein the skirt comprises of elastomeric material.

12. The improved ocular surgical docking system of claim 1 further comprising at least one lightning mechanisms coupled to the docking device or gonioprism.

13. The improved ocular surgical docking system of claim 3, wherein once the vacuum seal is created, the docking skirt is fixed at the docking location.

14. The improved ocular surgical docking system of claim 1, wherein, during a medical procedure, the proximal lens provides visualization of an anterior chamber of an eye where the cornea and the iris join at an area that is not otherwise visible when looking directly at the eye.

15. The improved ocular surgical docking system of claim 1, wherein the docking base is coupled with a gonioprism using a screwing mechanism.

16. The improved ocular surgical docking system of claim 1, wherein the docking base is coupled with a gonioprism using a latching mechanism.

17. The improved ocular surgical docking system of claim 1, wherein the gonioprism comprises titled walls.

18. The improved ocular surgical docking system of claim 1, wherein the tilted walls comprise at least optical device, such as a mirror, that refracts or reflects light.

19. A method for improved ocular surgical docking, comprising:
providing a docking base;
coupling a docking skirt to a distal end of the docking base;
coupling a gonioprism to the docking base; and
fixing the docking skirt to a docking location.

20. The method for providing an improved ocular surgical docking of claim 19, fixing the docking skirt to a docking location comprises operably coupling a vacuum hose to the docking base and, using the vacuum hose, removing fluid from an interior volume of the docking skirt placed at a docking location, such that the skirt creates a vacuum seal at the docking location.
